(19) United States
(12) Patent Application Publication
(10) Pub. No.: US 2006/0051290 A1
(43) Pub. Date: Mar. 9, 2006

(54) Short carbon nanotubes as adsorption and retention agents

(75) Inventors: Lon J. Wilson, Houston, TX (US); Michael G. Rosenblum, Sugar Land, TX (US)

Correspondence Address: CONLEY ROSE, P.C., P. O. BOX 3267, HOUSTON, TX, 77253-3267, US

(73) Assignee: William Marsh Rice University, Houston, TX (US)

(21) Appl. No.: 11/180,088

(22) Filed: Jul. 13, 2005

Related U.S. Application Data
(60) Provisional application No. 60/587,344, filed on Jul. 13, 2004.
(60) Provisional application No. 60/626,062, filed on Nov. 8, 2004.
(60) Provisional application No. 60/629,498, filed on Nov. 19, 2004.

(51) Int. Cl. A61K 51/00; A61N 5/00

(52) U.S. Cl. 424/1.11; 600/1

(57) ABSTRACT

A method for delivering a radiotherapeutic agent to a target, comprises administering a composition comprising water-soluble nanotubes having an average length less than 50 nm and a radionuclide so as to expose the target to the composition. The nanotubes can be functionalized with a monoclonal antibody having an affinity for the target. The radionuclide can be contained in the nanotubes, which can be derivatized. The nanotubes can be loaded with I₂ or ²¹¹AtI, another α-emitter, including but not limited to ²¹¹AtI, ²²⁵Ac, ²¹²Bi, ²¹³Bi, and combinations thereof. The nanotubes have an average length less than 40 nm, or an average length less than 30 nm.

### CROSS-REFERENCE TO RELATED APPLICATIONS

This non-provisional application claims the benefit of U.S. Provisional Application No. 60/587,344, filed Jul. 13, 2004, which is hereby incorporated by reference in its entirety. The present application further claims benefit of Ser. No. 60/626,062 filed Nov. 8, 2004, and Ser. No. 60/629,498, filed Nov. 19, 2004, both of which are incorporated by reference.

### STATEMENT REGARDING FEDERALLY SPONSORED RESEARCH OR DEVELOPMENT

Not applicable.

### FIELD OF THE INVENTION

This invention relates to the field of nanotubes and more specifically to shortened nanotubes containing magnetic nanomaterials.

### BACKGROUND OF THE INVENTION

Metals and radioisotopes have been used as the active components in contrast agents in such medical uses as magnetic resonance imaging and x-ray imaging. In such uses, the metals and radioisotopes are placed in the body. Drawbacks to placing such metals and radioisotopes in the body include their toxicity. Molecules such as chelators have been developed to overcome such drawbacks. The chelators typically contain the metals and radioisotopes and regulate their toxicity. Drawbacks to using chelators include each metal and radioisotope typically requiring a unique chelator. In some instances, the chelators are developed over years of tests and research.

Magnetic contrast agents typically increase the relaxation rates of protons in surrounding water, which may enhance the detected magnetic resonance signal in tissue. This effect may be used to increase the relative differences of relaxation times in adjacent tissues (which may otherwise be quite small), thereby raising the resolution and sensitivity of the magnetic resonance imaging technique. For instance, molecular contrast agents that have been studied are coordination complexes of the Gd(III) ion, which with its seven unpaired f-electrons has a very high paramagnetic moment as well as a favorable electron spin relaxation time.

A goal of contrast agent development is to increase the inherent relaxation potency offered by agents. The quantitative measure of relaxation effect is called relaxivity, which is a characteristic measure of a material's ability to change water proton relaxation times. Relaxivity increases typically boost the contrast an agent provides while also lowering the dosage required for imaging. An additional goal is to raise relaxivities to the levels needed for imaging individual cells and receptor sites. Drawbacks to conventional contrast agents include their lack of sufficient relaxivities to achieve such goals.

Consequently, there is a need for improved contrast agents. Moreover, needs exist for contrast agents having reduced toxicity to the body. Further needs include contrast agents that can be used without unique chelators. Additional needs include a contrast agent with increased relaxation potency.

### BRIEF SUMMARY OF SOME OF THE PREFERRED EMBODIMENTS

These and other needs in the art are addressed in one embodiment by a shortened carbon nanotube comprising a length of about 100 nm or less and further comprising a cargo.

In another embodiment, these and other needs in the art are addressed by a method for preparing a contrast agent. The method comprises providing a shortened carbon nanotube having a length of about 100 nm or less. In addition, the method includes filling at least a portion of the shortened carbon nanotube with a cargo. The method further includes derivatizing the shortened carbon nanotube.

In other embodiments, the present invention provides: 1) short carbon nanotubes that retain or more other chemical species, 2) a process for retention of other species by short carbon nanotubes, 3) a process for carrying out a chemical reaction involving a species retained by a short carbon nanotubes segment, 4) devices and applications using the items above, including: a) chelating agents, b) radiotherapeutic pharmaceutical compositions, and c) a method and composition for storing hydrogen

According to certain embodiments, short segments of carbon are converted into a composition containing both the nanotube segments and desired retained chemical species, where the retained chemical species are difficult to remove from the nanotubes. It is believed that the difficulty of removal of these retained species is due to their presence in the hollow space at the center of the nanotube. As mentioned herein, this present invention makes use of with short nanotube segments. Such segments are usually less than 200 nanometers (nm) long. More useful segments are less than 100 nm long and the preferred samples are predominantly in the length range between 20 and 50 nm.

The foregoing has outlined rather broadly the features and technical advantages of the present invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a detailed description of the preferred embodiments of the invention, reference will now be made to the accompanying drawings in which:

FIG. 1 illustrates an NMRD profile of Gd³⁺ shortened carbon nanotubes compared to [Gd(DTPA)]²⁻;

FIG. 2 illustrates an XRD powder pattern of Gd³⁺ shortened carbon nanotubes; and

FIG. 3 illustrates Gd4d_{s/2} x-ray photoelectron spectra of Gd³⁺ shortened carbon nanotubes, GdCl₃, and Gd₂O₃.

### DETAILED DESCRIPTION OF THE PREFERRED EMBODIMENTS

In an embodiment, a contrast agent comprises a shortened carbon nanotube containing a cargo. Without being limited by theory, the shortened carbon nanotube acts as a carbon coating that does not interfere with the fundamental properties of interest that the cargo contains. Further, without being limited by theory, the shortened carbon nanotube may also at least partially shield the body from the toxicity of the cargo. In some embodiments, the shortened carbon nanotube is derivatized. For instance, the shortened carbon nanotube may be derivatized to water-solubilize the nanotube. In an embodiment, the contrast agent is prepared by a method comprising cutting or shortening the carbon nanotubes, filling the shortened carbon nanotubes, and derivatizing the shortened carbon nanotubes. In an alternative embodiment, the shortened carbon nanotubes are derivatized before being filled.

Carbon nanotubes refer to a type of fullerene having an elongated, tube-like shape of fused five-membered and six-membered rings. Carbon nanotubes can be single walled carbon nanotubes (SWNTs) or multi-walled carbon nanotubes. Single-walled carbon nanotubes differ from multi-walled carbon nanotubes by the number of tubes. For instance, single-walled carbon nanotubes have one tube about a given center, and multi-walled carbon nanotubes comprise at least two nested tubes about a common center.

Carbon nanotubes may be of any size. Typically, carbon nanotubes are of micron-length. Shortened carbon nanotubes refer to carbon nanotubes that have reduced length. For instance, shortened carbon nanotubes have a length of about 100 nm or less, alternatively of about 50 nm or less, and alternatively from about 20 nm to about 50 nm.

The shortened carbon nanotubes may be prepared from typical carbon nanotubes by any suitable method. Without limitation, examples of suitable methods include the fluorination-cutting process, acid treatment, oxidation, and the like. The fluorination-cutting process is disclosed in Gu et al., Nano Letters, pgs. 1,009-1,013 (2002) and U.S. Patent Publication No. 2004/0009114 A1, which are each incorporated by reference herein in their entirety. For instance, in the fluorination-cutting process, the carbon nanotube is cut by reacting with a fluorinating agent. The process includes heating the full-length carbon nanotube to a suitable temperature from about 30° C. to about 200° C., alternatively about 50° C. The carbon nanotube is heated for a time from about 0.5 hours to about 3 hours, alternatively about two hours. The carbon nanotube is heated in a fluorine atmosphere. For instance, the atmosphere may include 1% fluorine in helium. The fluorinated carbon nanotubes may then be heated at a suitable temperature (e.g., 1,000° C.) for a suitable time (e.g., from about 1 to about 4 hours) under an argon atmosphere in a temperature-programmable furnace such as a quartz tube furnace. This process may cut the typical, long carbon nanotubes into the shortened carbon nanotubes. In an embodiment, the shortened carbon nanotubes are exposed to a high vacuum to remove any traces of gases. For instance, cutting of the carbon nanotubes may generate small amounts of CF₄ along with traces of COF₂ and CO₂. Without being limited by theory, the fluorine gas flows around the carbon nanotube and CF bonds attach in non-uniform bands on the surface and/or inside of the carbon nanotubes. By the process above, the carbon nanotubes may be cut at the CF bands. Further, without being limited by theory, the fluorine gas flows around the carbon nanotube, and CF bonds attach in spots on the surface of the carbon nanotube. By the process above, the CF spots may volatize and create holes in the walls of the carbon nanotubes (e.g., side wall defects).

In some embodiments, the fluorination-cutting process may also remove at least a portion of residual iron catalyst particles in the carbon nanotube. For instance, the typical commercial nanotubes (e.g., those produced by Fe(CO)₅-catalyzed decomposition of CO at high temperature and pressure) are ca. 98% free of residual iron particles. Without being limited by theory, any remaining traces may interfere with the magnetic and relaxivity characterization of the contrast agent. Further, without being limited by theory, fluorination may damage the structures of the remaining trace amounts of iron-containing catalyst particles, allowing their elimination by aqueous acid extraction.

In an embodiment, the shortened carbon nanotubes may be filled with a cargo. In some embodiments, the cargo may include magnetic material, molecular iodine, metal salt, metal salt hydrate, metal oxide, or combinations thereof. The magnetic material may include an iron oxide, a magnetic metal, a magnetic metal salt, a magnetic metal salt hydrate, a magnetic metal oxide, or combinations thereof. Without limitation, examples of suitable iron oxides include Fe₂O₃ and Fe₃O₄. In addition, without limitation, examples of suitable magnetic metals include gadolinium, nickel, cobalt, holmium, or combinations thereof. Examples of a magnetic metal salt include, without limitation, gadolinium halides such as fluoride, chloride, bromide, and iodide; oxides; nitrates; hydroxides; acetates; citrates; sulfates; phosphates; their hydrates; or combinations thereof. For instance, non-limiting examples of suitable magnetic metal salts include GdCl₃. Gd(NO₃)₃, FeCl₃, Fe(NO₃)₃, NiCl₂, CoCl₂, CoCl₃, or combinations thereof. Further, without limitation, examples of suitable magnetic metal salt hydrates are hydrates of such magnetic metal salts. It is to be understood that all or a portion of the nanotube may be filled. The shortened carbon nanotubes may be filled through the ends of the shortened carbon nanotubes and/or through the side wall defects. The shortened carbon nanotubes may be filled by any suitable method. In one embodiment, the shortened carbon nanotubes may be filled by a method including generating a well-dispersed nanocapsule suspension in water by vigorous stirring and brief immersion in an ultrasonic bath. An aqueous iron nitrate solution may then be added, and the mixture stirred for at least one hour. The mixture is centrifuged to remove the filled shortened carbon nanotubes, which may then be rinsed with excess distilled water and vacuum dried. The magnetic material may be converted to an oxide by calcination, which may be conducted by heating the filled shortened carbon nanotubes gradually (e.g., <5° C. per min) in a stream of argon at a suitable temperature for a suitable time. In an embodiment, a suitable temperature is from about 100° C. to about 1,200° C., alternatively 450° C.; and a suitable time is from about 1 hour to about 10 hours, alternatively 5 hours. The heating is followed by cooling under vacuum. In some embodiments, following formation of the oxide (e.g., gadolinium oxide), the filled shortened carbon nanotubes are reduced with hydrogen gas at elevated temperatures (excluding oxygen in the apparatus) to form encapsulated metal (e.g., gadolinium metal).

In an alternative embodiment, the shortened carbon nanotubes are filled with cargo by inserting liquid metals and/or molten salts. For instance, shortened carbon nanotubes may be filled by immersing them in liquid metals or molten (melted) salts directly. Without being limited by theory, the filling may occur via capillary action. Examples of filling nanotubes are disclosed in Chen et al., “Synthesis of carbon nanotubes containing metal oxides and metals of the d-block and f-block transition metals and related studies,” J. Mater. Chem., 7, 545-549 (1997) and Brown et al., “High yield incorporation and washing properties of halides incorporated into single walled carbon nanotubes,” Appl. Phys. A 76, 457-462 (2003), which are incorporated by reference herein in their entirety.

Without being limited by theory, the filling mechanism of the shortened carbon nanotube may involve capillary action. Further, without being limited by theory, a strong interaction between the cargo and the interior sidewall of the shortened carbon nanotube may drive the filling and retain the contents in place.

In some embodiments, the filled shortened carbon nanotubes may have the encapsulation of the magnetic material verified, and the lack of leaking of the encapsulated contents verified. Without limitation, verification may be accomplished by energy dispersive X-ray fluorescence, electron microscopy, inductively coupled plasma-atomic emission spectroscopy, and the like. For instance, rapid metal assay may be done using EDS elemental analysis (energy dispersive X-ray fluorescence, operating in conjunction with a scanning electron microscope (SEM)). High-resolution electron microscopy imaging may also be used to characterize the contents of filled shortened carbon nanotubes. In one embodiment, for quantifying potential metal content leaching into water, filled shortened carbon nanotubes may be suspended in aqueous solutions over a large pH range for different lengths of time, after which the nanotubes may be separated by centrifugation. The cargo may be quantitatively assayed by the filled shortened carbon nanotubes being digested in hot nitric acid, followed by metal quantification using an inductively coupled plasma (e.g., atomic emission spectrometer). The cargo may then be compared to any metal content in the aqueous supernatant. Without being limited by theory, such a method may reveal the propensity, if any, for the filled nanocapsules to leak their metal contents over a wide range of pH. In other embodiments, an independent test for metal loss when the contents are gadolinium (Gd) may involve relaxivity measurements. For instance, if free Gd(III) is released by the filled shortened carbon nanotubes while in water, these ions may have a measurable relaxation effect on the supernatant water protons. If the measured relaxivity of the supernatant decreases upon addition of the ligand H₆TTHA (e.g., H₆TTHA is triethylenetetramine-N,N,N′,N″,N′″N′″-hexacetic acid), this may indicate that free Gd(III) may be present because the strongly-bound Gd-TTHA complex has no inner-sphere water molecules and a much lower relaxivity than that of free Gd(III)_{aq}.

In an embodiment in which at least one end of the filled shortened carbon nanotube is open, the filled shortened carbon nanotube may have the open end or ends sealed or closed by any suitable method. Without limitation, examples of suitable methods include chemical methods, thermal methods, and the like. An example of a chemical method includes constructing a chemical barrier across the open tube ends. Intramolecular bond formation performed by cross metathesis on olefin groups attached to the tube ends with an organometallic ruthenium compound may cover the tube ends with covalently cross-linked groups. In an embodiment, the tube ends may be oxidized to carboxylate groups. An example of such an oxidation is disclosed in Chen et al., “Solution Properties of Single-Walled Carbon Nanotubes,” Science 282, pgs. 95-98 (1998). SOCl₂ may convert the carboxylates to acid chloride groups, which may then be reacted with substituted amines to form amides. In an embodiment in which the amine groups have unsaturated ethylene moieties at the ends, the moieties may be covalently linked together with the organometallic ruthenium compound (e.g., Grubb's catalyst). Without being limited by theory, entropic and steric considerations may promote intramolecular bond formation as opposed to interparticle linking by the catalyst. An example of a thermal method includes annealing the shortened carbon nanotube. For instance, the ends of the shortened carbon nanotube may be thermally annealed to form hemispherical carbon domes or end caps that may seal the interior contents in place. In some embodiments, the annealing may occur at temperatures from about 100° C. to about 1,500° C., alternatively at about 1,000° C. The annealing may occurring for any suitable duration. In an embodiment, annealing may occur from about 1 hour to about 12 hours. In an embodiment, the chemical method may be followed by the annealing method.

In some embodiments, the filled shortened carbon nanotubes may be derivatized for any desired purposed such as biocompatibility, water solubility, disease targeting, organ targeting, in vivo half life, interparticle clustering, and the like. In an embodiment, the filled shortened carbon nanotubes may be derivatized for water solubility. Without being limited by theory, attaching water-solubilizing groups may impart needed solubility to the filled shortened carbon nanotube surfaces and promote biocompatibility. In addition, groups that hydrogen-bond to solvent waters may also promote enhanced relaxivity with a large surface area for close interaction. Derivative groups may also be used to link targeting and other desirable moieties to the filled shortened carbon nanotube for advance contrast applications.

In an embodiment, the filled shortened carbon nanotubes may be water-solubilized via exterior sidewall covalent derivatization. Exterior covalent derivatization may be accomplished by any suitable method. An example of a suitable method is addition chemistry. Without being limited by theory, addition chemistry includes formation of new bonds between the carbons of the nanotube sidewalls and substituents. Without limitation, examples of substituents include carbon, oxygen, nitrogen, halogens, lithium, transition metals, boron, silicon, sulfur, phosphorus, hydrogen, and the like. In addition, after first adding groups or atoms, substitution reactions may be used to further modify nanotube surfaces. For example, fluorinated tubes may be hydroxylated to form carbon-oxygen bonds. Alternatively, if there are holes and/or oxygenated portions on the nanotube surface arising from their production or handling (including carbonyls, carboxylates, hydroxyl groups, and/or hydrogen), corresponding addition or substitution reactions may occur with such groups.

Without limitation, an example of addition chemistry includes addition of substituents across carbon-carbon double bonds. For instance, the 1,3-dipolar cycloaddition of azomethine ylides may be used. The 1,3-dipolar cycloaddition of azomethine ylides may be formed from heating aldehydes and amino acids. The 1,3-dipolar cylcoaddition may covalently link poly(ethylene glycol) moieties. Without being limited by theory, poly(ethylene oxide) fragments linked to shortened carbon nanotubes by this method may provide the shortened carbon nanotubes with solubility and without intermolecular aggregation. This reaction is the 1,3-dipolar cycloaddition of azomethine ylides. These intermediate species may be produced by reaction of an aldehyde with an amino acid. Cycloaddition for full-length (standard) single-walled carbon nanotubes is disclosed in Georgakilas et al., “Organic Functionalization of Carbon Nanotubes,” J. Am. Chem. Soc. 2002; volume 124, pages 760-761, which is incorporated by reference herein in its entirety. In an embodiment, carboxylic and poly(ethylene oxide) groups may be included to introduce water-solubilizing and biocompatible functional groups. Such groups may be added either first as substituents of the cycloaddition reagents, or later linked to the groups attached in the initial surface cycloaddition. Useful alternative derivatizations include base-induced cycloaddtion of bromomalonates for introducing carboxylate functionalities, Diels-Alder cycloadditions, radical additions and fluorination followed by nucleophilic replacement of fluorine addends. In some embodiments, longer chain PEO groups or serinol derivatives may be employed to enhance water solubility.

In an embodiment, the filled shortened carbon nanotubes may be derivatized for biocompatibility. For instance, derivatizing for biocompatibility may include providing a non toxic or reduced toxic nanotube. Toxicity refers to the capability for damaging or injuring the body. In an embodiment, chemical groups such as without limitation carboxylates, poly(ethylene oxide) fragments, hydroxyls, and/or amino groups may be used to reduce toxicity. Such groups may be linked to the filled shortened carbon nanotubes by addition chemistry.

In some embodiments, the filled and shortened carbon nanotubes may be characterized by any suitable method for separation. Without limitation, methods for separation include differentiating according to size, content, and/or derivatization motif. For instance, purification may include high-performance liquid chromatography (HPLC) with a size-exclusion chromatography (SEC) column to generate size-separated fractions of nanocapsules with narrow size distributions.

The contrast agents comprise high relaxivities. Relaxivity refers to the measure of the ability of a particular substance to change the proton relaxation time of water molecules. The contrast agents may have relaxivites from about 5 mM⁻¹ s⁻¹ to about 1,500 mM⁻¹ s⁻¹, alternatively from about ₅ mM⁻¹ s⁻¹ to about 150 mM⁻¹s⁻¹.

The contrast agents may be used in any suitable imaging medium such as MRI and x-ray. For instance, the cargo may include gadolinium when the use is to be as an MRI contrast agent, and the cargo may include molecular iodine when the use is to be as an x-ray contrast agent. In some instances, the contrast agents may have multi-modal usage (e.g., may be used in both MRI and x-ray imaging). For instance, a gadolinium filled tube may be used for both MRI and x-ray imaging, or a tube filled with a mixture of gadolinium and iodine (as elements, compounds and/or as a binary salt (gadolinium iodide)) may be made for a multi-modal contrast agent.

To further illustrate various illustrative embodiments of the present invention, the following examples are provided.

### EXAMPLE 1

This example indicated the high relaxivities of filled shortened carbon nanotubes. Cut nanotubes were filled with two different magnetic compounds, iron oxide and gadolinium(III) chloride. The metal contents were determined by inductively coupled plasma (ICP), and r-values (e.g., relaxivity) were calculated on a metal content basis. The measured r₁ value (e.g., 0.47 T, 40° C.) for iron-oxide filled nanocapsules (derivatized with a simple hydroxylation process such as with the Fenton reaction (e.g., H₂O₂+Fe²⁺→.OH @ pH 3-5) in water was about 40 mM⁻¹s⁻¹. In addition, gadolinium chloride filled nanocapsules, not derivatized but suspended in water with the aid of a surfactant such as sodium dodecylbenzene sulfate, displayed an r₁ value of about 150 mM⁻¹s⁻¹. The results are indicated in Table I below. In the table, T1 refers to the longitudinal relaxation time of water protons.

TABLE I

| Nanocapsules filled with: | T₁/ms | metal conc./mg L⁻¹ | r₁/mM⁻¹ s⁻¹ |
|---|---|---|---|
| iron oxide | 174.5 | 7.5 | 41.4 |
| gadolinium chloride | 182.6 | 5.8 | 144.9 |

### EXAMPLE 2

Shortened carbon nanotubes were explored as nanocapsules for MRI-active Gd³⁺ ions. The shortened carbon nanotubes were loaded with aqueous GdCl₃, and characterization of the resulting Gd³⁺ showed increased relaxivities.

The long carbon nanotubes used were produced by the electric arc discharge technique with Y/Ni as the catalyst. The long carbon nanotubes were cut into shortened carbon nanotubes by fluorination followed by pyrolysis at 1,000° C. under an inert atmosphere. The shortened carbon nanotubes were then loaded by soaking and sonicating them in HPLC grade DI water (pH=7) containing aqueous GdCl₃.

To load the shortened carbon nanotubes, 100 mg of shortened carbon nanotubes and 100 mg of anhydrous GdCl₃ were stirred together in 100 ml deionized HPLC grade water and sonicated in a 30 W batch sonicator for 60 minutes. The solution was left undisturbed overnight, whereupon the Gd³⁺ loaded shortened carbon nanotubes flocculated from the solution. The supernatant solution was then decanted off. The sample was then washed with 25 ml of fresh deionized HPLC grade water and batch sonicated to remove any unabsorbed GdCl₃. The Gd³⁺ loaded shortened carbon nanotubes flocculated from the solution, and the supernatant solution was removed by decantation. The procedure was repeated three times. Multiple samples were prepared to demonstrate reproducibility. The sample was air dried, and an ICP analysis performed showed the Gd content to be 2.84% (m/m).

The relaxivity of the Gd³⁺ loaded shortened carbon nanotubes was measured. For the relaxivity measurements, a saturated solution of 40 mg of the Gd³⁺ loaded shortened carbon nanotubes in 20 ml of a 1% sodium dodecyl benzene sulfate (SDBS) aqueous solution and another of 10 mg of the Gd³⁺ loaded shortened carbon nanotubes in 5 ml of a 1% biologically-compatible pluronic F98 surfactant solution were prepared. 10% of the Gd³⁺ loaded shortened carbon nanotubes dispersed and formed a stable suspension. These two supernatant (suspensions) solutions were then used for the relaxometry experiments.

Single-point relaxation measurements were performed on the Gd³⁺ loaded shortened carbon nanotubes with controls at 60 MHz/40° C. The longitudinal relaxation rates (R₁) were obtained by the inversion recovery method at pH=7.0, and the longitudinal relaxivity (r₁) was obtained by (T₁⁻¹)_{obs}=(T₁⁻¹)_{d}+r₁[Gd³⁺], where T_{1obs} and T_{1d} are the relaxation times in seconds of the sample and the matrix (aqueous surfactant solution), respectively, and [Gd³⁺] is the Gd concentration in mM. The absence of free (non-encapsulated) Gd³⁺ ion in the sample was confirmed by measuring the proton relaxivities of the solutions at 60 MHz before and after the addition of the ligand, TTHA⁶⁻ (pH=7). This ligand TTHA⁶⁻ typically forms a highly stable complex with Gd³⁺, which contains no inner-sphere water molecule. Therefore, [GdTTHA]³⁻ with no inner-sphere water molecule has a lower relaxivity than (Gd³⁺ —OH₂) centers, and therefore any decrease in relaxivity observed upon addition of TTHA⁶⁻ may signal the presence of free Gd³⁺ ion. For both solutions, the relaxation rates with and without TTHA⁶⁻ were identical, which implied the absence of accessible (exo shortened carbon nanotubes) aquated Gd³⁺ ions.

After completion of the relaxation rate measurements, the Gd-content of the sample solution was determined by ICP to calculate the relaxivity. The results are shown in Table II. In preparation for the ICP measurements, the solutions were treated with cc. 90% HNO₃ and heated until a solid residue was obtained. They were then treated with a a 30% H₂O₂ solution and heated to completely remove any remaining carbonaceous material. This solid residue was dissolved in 2% HNO₃ and analyzed by ICP. ICP analysis was performed on an inductively coupled atomic emission spectrometer with a CCD detector. For conditions, Gd lines at 335.05 nm, 342.35 nm, and 376.84 nm were initially chosen. Seven scans were performed for each sample (relative standard deviation=0.2%). The Gd line at 376.84 showed a higher intensity and was chosen for the final Gd concentration. Sc (λ=361.38 nm) was used as the internal drift standard.

Apart from the presence of Gd, the ICP analysis also showed 0.1 to 0.5 ppm of Ni present as impurity, but Y was not detected within the limits of the instrument (1 ppb). The large T₁ values of the unloaded shortened carbon nanotubes demonstrate that the presence of the Ni in the sample has no influence on the relaxation rates.

Upon completion of the relaxation rate measurements, the Gd-content of the sample solution was determined by ICP-OES to calculate the relaxivity. The results of the relaxation rate measurements and relaxivity calculations are given in Table II.

TABLE II
Proton relaxivities, r₁, (mM⁻¹ s⁻¹) of various
sample solutions at 60 MHz and 40° C.

| | C_{Gd} | C_{Gd} | T₁ | R₁ | R_{1d} | r₁ |
|---|---|---|---|---|---|---|
| Sample | (ppm) | (mM) | (ms) | (s⁻¹) | (s⁻¹) | (mM⁻¹ s⁻¹) |
| Gd³⁺ₙ shortened | 7 | 0.044 | 127.3 | 7.85 | 0.25 | 173 |
| tubes^{a} | | | | | | |
| Gd³⁺ₙ shortened | 7.8 | 0.049 | 120.6 | 8.29 | 0.24 | 164 |
| tubes^{b} | | | | | | |
| Shortened tubes | — | — | 2050 | 0.48 | 0.25 | — |
| [Gd(H₂O)₈]³⁺ | 313 | 1.99 | 59.0 | 16.95 | 0.24 | 8.4 |

^{a}1% SDBS surfactant solution.
^{b}1% pluronic F98 surfactant solution.

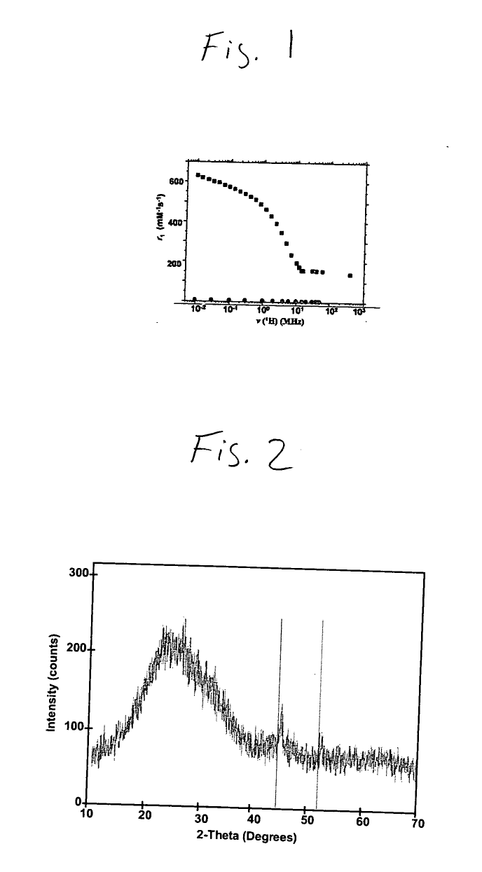

As shown in the table, the Gd³⁺ₙ shortened carbon nanotubes significantly reduced the relaxation rates relative to pure surfactant solution or unloaded shortened tubes. Comparing the relaxivity values of the Gd³⁺ₙ shortened carbon nanotube sample with [Gd(H₂O)₈]³⁺, the r₁ of aquated Gd³⁺ is 20 times lower at 60 MHz/40° C. than for the Gdₙ³⁺ shortened carbon nanotube. Thus, the relaxivity obtained for the Gd³⁺ₙ shortened carbon nanotube sample of r₁ 170 mM⁻¹ s⁻¹ is nearly 40 times greater than any current Gd³⁺-based oral or ECF CA, such as [Gd(DTPA)(H₂O)]²⁻ with r₁ 4 mM⁻¹ s⁻¹. It is also nearly 8 times greater than ultra small superparamagnetic iron oxide (USPIO) contrast agents with r₁ 20 mM⁻¹ s⁻¹. We observed small variability in the relaxivity values of different batches of Gd³⁺ₙ shortened carbon nanotubes and different surfactants used, but the order of magnitude reported in Table II was always the same (r₁ =159 mM⁻¹ s⁻¹ to 179 mM⁻¹ s⁻¹). The measurement of proton relaxivity for a Gd³⁺ₙ shortened carbon nanotube sample in 1% SDBS solution as a function of the magnetic field is presented in FIG. 1. This Nuclear Magnetic Relaxation Dispersion (NMRD) profile was recorded for an aqueous solution of Gd³⁺ₙ shortened carbon nanotubes in a 1% SDBS solution at 37° C. Also presented, for comparative purposes, are data for one of the commercially-available MRI CAs, [Gd(DTPA)(H₂O)]²⁻, presently in clinical use. As shown, for any magnetic field in FIG. 1, the relaxivity for the Gd³⁺ₙ shortened carbon nanotubes is remarkably larger than for the clinical CA. This is true at the standard MRI field strength (nearly 40 times larger) for clinical imaging of 20-60 MHz (170 mM⁻¹ s⁻¹ vs. 4.0 mM⁻¹ s⁻¹), but is even more pronounced (nearly 90 times larger) at very low fields such as 0.01 MHz (635 mM⁻¹ s⁻¹ vs. 7.0 mM⁻¹ s⁻¹).

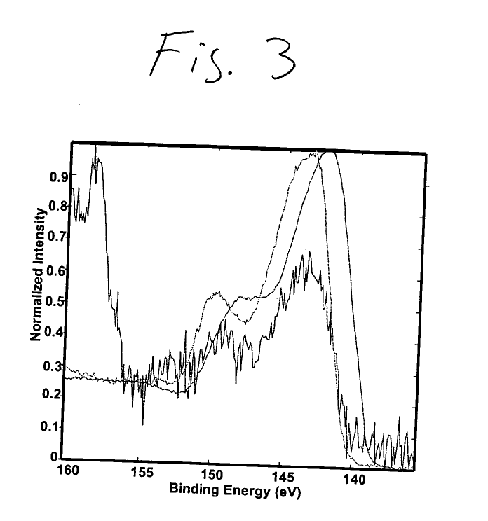

FIG. 2 illustrates an XRD pattern of a Gd³⁺ₙ shortened carbon nanotube. X-ray powder diffraction (XRD) was performed using a diffractometer with a Cu target. The scanning was from 10° to 70° at 0.04°/step. As shown, FIG. 2 indicates two small peaks from carbon, with no diffraction peaks due to crystalline Gd³⁺-ion centers. An XPS spectrum (x-ray photoelectron spectra) of a Gd³⁺ₙ shortened carbon nanotube is shown in FIG. 3. An XPS instrument was used with photo-emissions produced via a monochromatic Al K_{α} x-ray source (1486.6 eV) operated at 350 W. Photo-emissions were acquired at a take off of 45° as defined relative to the surface plane. These were passed through a hemispherical analyzer operated in the fixed retard ratio mode at a pass energy of 11.75 eV. Curve fitting and quantification were accomplished following the application of a Shirley background subtraction routine. The XPS spectrum shown in FIG. 3 demonstrates the presence of Gd³⁺ in the sample, and further comparisons with commercial anhydrous GdCl₃ and Gd₂O₃ samples in FIG. 3 demonstrate that the confined Gd³⁺-ion clusters more closely resemble GdCl₃₊. Thus, the absence of any Gd³⁺-ion crystal lattice detectable by XRD may be attributed to the small cluster size (1 nm×2-5 nm), the low gadolinium content (2.84% (m/m) from ICP) and/or the amorphous nature of the hydrated Gd³⁺ₙ-ion clusters with their accompanying Cl⁻ counterions (Gd Cl ratio 1 3 by XPS).

This invention also includes a method for performing a chemical reaction with a species retained by a short carbon nanotubes. The site of retention may be at the end, side, a defect site, or interior of the short nanotube. Specifically, the reaction may be an oxidation/reduction reaction or a catalytic reaction. Alternatively, it may be an ion-exchange reaction where an ion associated with the nanotubes is exchanged for another. The reaction may be one in which electronic charge is exchanged between the reagent species. As demonstrated in the Example, such reactions may convert the retained species to one with a different retention half-life.

### EXAMPLE 3

We have established that Iodine⁻¹²⁵ is effectively retained by short carbon nanotubes. In an environment where nanotube/iodine⁻¹²⁵ complexes are subjected to continuous rinsing, short nanotubes exhibit retention times for iodine⁻¹²⁵ that are one to two orders of magnitude longer than those for other carbonaceous materials, including as-produced carbon nanotubes, fluorinated carbon nanotubes, C₆₀, graphite, and fluorinated graphite. In all these cases, it is known that the iodine associates with the carbon material as a negative ion. It is further demonstrated that the I⁻ associated with the short nanotubes can easily be converted to I₂ by an oxidation-reduction reaction when the nanotubes/iodine complex is exposed to hydrogen peroxide. The neutral I₂ molecule has an even longer half-like of retention by the short nanotubes than the parent I⁻ ion.

We compared the wash-off rates for cut SWNTs to those for uncut SWNTs and to charcoal. Using ¹²⁵I⁻ adsorbed on/retained in the carbonaceous species, we obtained the following Table:

| | t_{1/2}, hours | | |
|---|---|---|---|
| Adsorbent | Water | Aqueous NaI soln | Aqueous H₂O₂ soln |
| Cut SWNTs | 2720 | 422 | 14300 |
| Uncut SWNTs | 54 | 14 | 147 |
| Charcoal | 81 | 14 | 309 |

The rate of ¹²⁵I⁻ wash-off by the NaI solution was found to be 4-7 times higher than for pure water, which can be explained by the equilibrium competition between I⁻ from NaI and ¹²⁵I⁻ from Na¹²⁵I (common ion effect). The further dramatic decrease in the desorption rate caused by an aqueous H₂O₂ solution is attributed to the oxidation of I⁻ to I₂ by H₂O₂, with I₂ having a much greater tendency to adsorb to (and likely within) cut-SWNTs than the I⁻ ion. Oxidation of I⁻ to I₂ within a cut-SWNT is one of the first documents examples of the chemical reaction occurring within the confines of the of a carbon nanotube structure. As shown above, it was found that cut-SWNTs have the slowest ¹²⁵I⁻ release rate of any of the carbonaceous materials. It is expected that this level of retention will hold true for other radioactive materials and/or therapeutic agents. Because the NaI solution was so much more effective than water or peroxide for removing the radioactive species from the SWNT, it is further contemplated that such a system could be used when it is desirable to remove one or more radioactive agents or other retained compound from a nanotube composition. This may be the case when it is desired to dispose of or recycle either the nanotubes or the radioactive agent, or when it is desired to replace one radioactive agent with another. Hence, a method for removing a retained compound from a nanotube may comprise washing the agent-containing nanotube with a solution of a salt of an isotope of the radiotherapeutic agent. The isotope may be the same (radioactive) isotope or a different isotope.

One application of the radiotherapeutic agents retained in cut nanotubes is in radiotherapy. Historically, radiotherapy has been carried out by preparing a first compound comprising a targeting agent (such as a monoclonal antibody) and a chelating agent (which bonds to metal atoms), preparing a second compound comprising radioactive metal atoms, and mixing the first and second compounds together to form complexes comprising both the radioactive atom and the targeting agent. This mixture provides the therapeutic dose for a patient. One problem in targeted radiotherapy has been that there are few, if any, effective chelating agents for α-emitters like At⁻²¹¹ and Ac⁻²²⁵, and α-emitters are a very promising form of radiotherapeutic agent. As we have shown, short nanotubes are excellent chelating agents for large atoms. This invention provides short nanotubes that are associated with large atoms. The resulting compositions are radiotherapeutic compounds comprising short nanotubes, targeting agents, and radioactive species. These compounds may also contain a linking agent between the targeting agent and the short nanotube.

Beyond uses in radiotherapy, the present invention has important applications in hydrogen storage. It is known that certain metallic species readily bind with hydrogen. Such metals include magnesium, iron, titanium, and lanthanum, individually or in combination. Considerable research is underway to find an effective system for storage of hydrogen for a fuel, wherein the hydrogen is stored as a metallic hydride. It is desirable to control the kinetics and thermodynamics of hydrogen storage to make it reversible in a temperature range appropriate for use in vehicles and other hydrogen-fuel applications. Development of effective hydrogen storage compositions is enabled by supporting the metallic hydride-forming species on a structure with nanometer-scale dimensions. This invention provides a composition comprising short carbon nanotubes that retain metallic species. This composition is useful for storage and release of hydrogen.

In the example of a medical application such as that of the radiotherapeutic agent above, the long retention time in the nanotubes complex diminishes the toxicity of the retained species, because the radiotherapeutic agent is confined at the nanotube site and cannot interact chemically with body fluids. For effective sequestration of retained species, the endohedral form (where retained species reside inside the nanotube) of the present invention is particularly useful. The short nanotubes thus serves quite generally as a chelating agent, and is effective in any environment where one seeks to retain metal atoms by another chemical species. Toxic or radioactive waste sequestration are examples of such utility.

Possible uses for this invention include: 1) radiotherapeutic agents, 2) chelating agents, 3) vehicles for catalysis, 4) vehicles for hydrogen storage, and 5) radioactive and toxic waste removal agents.

Hence, one method for delivering a radiotherapeutic agent to a target comprises administering a composition comprising water-soluble nanotubes having an average length less than 50 nm and a radionuclide so as to expose the target to the composition. The nanotubes can be functionalized with a monoclonal antibody having an affinity for the target. The radionuclide can be contained in the nanotubes, which can be derivatized. The nanotubes can be loaded with I₂ or ²¹¹AtI, another α-emitter, including but not limited to ²¹¹AtI, ²²⁵Ac, ²¹²Bi, ²¹³Bi, and combinations thereof. The nanotubes have an average length less than 40 nm, or an average length less than 30 nm.

The present invention provides a carbon nanotube species (cut-nanotube) wherein the nanotube is vastly more effective in retaining other species than known forms of carbon nanotubes and other forms of carbon. The invention further provides a method for performing a chemical reaction with a species that is retained by a short carbon nanotube.

The steps involved in carrying out one embodiment of the method of the invention are: a) providing short carbon nanotubes, b) exposing the cut carbon nanotubes to an environment containing a form of the species desired to be retained by the nanotubes, and c) recovering the cut-nanotube/retained species complexes. The recovered nanotube/retained species complexes are useful in radiotherapy and can be derivatized with one or more targeting agents to increase their efficacy as therapeutic agents. In another embodiment of the invention, the steps include: a) providing at least one nanotube/retained species complex and b) exposing that complex to a reaction environment comprising at least one additional chemical species wherein the retained species undergoes a chemical reaction or serves as a catalyst for a chemical reaction.

Although the present invention and its advantages have been described in detail, it should be understood that various changes, substitutions and alterations may be made herein without departing from the spirit and scope of the invention as defined by the appended claims.

What is claimed is:

1. A method for delivering a radiotherapeutic agent to a target, comprising:
administering a composition comprising water-soluble nanotubes having an average length less than 50 nm and a radionuclide so as to expose the target to the composition.

2. The method according to claim 1 wherein said nanotubes are functionalized with a monoclonal antibody having an affinity for the target.

3. The method according to claim 1 wherein the radionuclide is adsorbed on the nanotubes.

4. The method according to claim 1 wherein the radionuclide is contained in the nanotubes.

5. The method of claim 1 wherein the nanotubes are derivatized.

6. The method of claim 1 wherein the nanotubes are loaded with I₂ or ²¹¹AtI.

7. The method of claim 1 wherein the radionuclide includes an α-emitter.

8. The method of claim 1 wherein the radionuclide includes an α-emitter selected from the group consisting of ²¹¹AtI, ²²⁵Ac, ²¹²Bi, ²¹³Bi, and combinations thereof.

9. The method of claim 1 wherein the nanotubes have an average length less than 40 nm.

10. The method of claim 1 wherein the nanotubes have an average length less than 30 nm.

11. A method for removing a radiotherapeutic agent from a nanotube, comprising:
washing the agent-containing nanotube with a solution of a salt of an isotope of the radiotherapeutic agent.
